(19) United States
(12) Patent Application Publication
(10) Pub. No.: US 2016/0216262 A1
(43) Pub. Date: Jul. 28, 2016

(54) Method for In Vitro Quantifying Allo-Antibodies, Auto-Antibodies and/or Therapeutic Antibodies

(71) Applicants: INSTITUT NATIONAL DE LA SANTE ET DE LA RECHERCHE MEDICALE, Paris (FR); UNIVERSITE PARIS DIDEROT (PARIS 7), Paris (FR); UNIVERSITE PARIS DESCARTES, Paris (FR); UNIVERSITE PIERRE ET MARIE CURIE (PARIS 6), Paris (FR); ASSISTANCE PUBLIQUE HOPITAUX DE PARIS, Paris (FR)

(72) Inventors: Marie-Agnes Dragon-Durey, Paris (FR); Marie Senant, Paris (FR)

(21) Appl. No.: 14/917,791

(22) PCT Filed: Sep. 11, 2014
(86) PCT No.: PCT/IB2014/064437

§ 371 Date: Mar. 9, 2016

(30) Foreign Application Priority Data
Sep. 12, 2013 (EP) 13306251.3

(51) Int. Cl. G01N 33/564; G01N 33/94; G01N 33/68

(52) U.S. Cl. CPC G01N 33/564; G01N 33/6854; G01N 33/94; G01N 2496/00

(57) ABSTRACT

An in vitro method for quantifying a target-specific test antibody in a test sample, comprising the steps of: a) performing an immunoassay using a target immobilized on a support which is brought into contact with the test sample, the immunoassay comprising a step of measuring the binding of the target-specific test antibody to the immobilized target by using a detectable non-antibody ligand that binds to the Fc region or to a light chain of an antibody, whereby a concentration-related value of the target-specific test antibody in the test sample is obtained, and b) comparing the concentration-related value obtained at step a) with a reference value obtained by performing an immunoassay using the target immobilized on a support which is brought into contact with a calibration sample comprising a known concentration of a target-specific calibration antibody, the immunoassay comprising a step of measuring the binding of the target-specific calibration antibody to the immobilized target by using the detectable non-antibody ligand of step a), and wherein: (i) the target-specific test antibody of step a) and the target-specific calibration antibody of step b) are identical, or (ii) the target-specific test antibody of step a) and the target-specific calibration antibody of step b) are distinct.

### FIELD OF THE INVENTION

The present invention relates to the field of quantification of a concentration of a target-specific test antibody that may be present in a test sample.

### BACKGROUND

Determining with accuracy a concentration of certain types of antibodies in samples may be of crucial importance in several medical conditions or for various scientific research purposes.

As a first example, it has been reported that allo-immunization may occur after that an organism has been exposed to a compound from an organism of the same species, usually after a transfusion or an allograft (transplantation). The receiving organism produces antibodies, namely allo-antibodies, against the components comprised in the transplant, and cases have even been reported wherein allo-antibodies are produced by a pregnant female against her own fetus.

Illustrative of allo-immunization, anti-IgA allo-antibodies may develop in patients who undergo an IgA deficiency and who are medically treated by administration of exogenous IgAs-containing compositions. These patients are currently allo-immunized after administration of blood-derived products. Selective IgA deficiency is the most frequent primary immunodeficiency in Europe and North America, with a prevalence estimated at 1/600 (Vorechovsky et al. Am. J. Hum. Genet. Genetic linkage of IgA deficiency to the major histocompatibility complex: evidence for allele segregation distorsion, parent-of-origin penetrance differences, and the role of anti-IgA antibodies in disease predisposition. 1999. 64:1096-1109). Most subjects with selective IgA deficiency are asymptomatic. Searching for the presence and the amount of anti-IgA antibodies is highly recommended for patients who have had adverse reactions or intolerance reactions during administration of blood products.

As a second series of examples, the well documented auto-immune diseases rely upon an immune response in an organism towards component(s) that naturally occur(s) in said organism. In auto-immune diseases, the organism produces auto-antibodies directed against its own components (organs, cells, proteins, carbohydrate, etc), which are recognized as non-self components by the organism. Among the auto-immune diseases, one may cite, among others, Graves' disease (Ploski et al. Current Genomics. The genetic basis of Graves' disease. 2011. 12; 542-563), rheumatoid arthritis (Lossius et al. Viruses. Epstein-Barr virus in systemic lupus erythematosus, rheumatoid arthritis and multiple sclerosis—association and causation. 2012. December; 4(12):3701-30), type I diabetes (Pihoker et al. Auto-antibodies in diabetes. Diabetes. 2005. December; 54 Suppl 2:S52-61), glomerulonephritis (Makker et al. Semin. Nephrol. Idiopathic membranous nephropathy: an autoimmune disease. 2011 Jul. 31(4):333-340), Sjögren syndrome (Selmi et al. J. Autoimmun. Primary biliary cirrhosis and Sjögren's syndrome: autoimmune epithelitis. 2012. August; 39(1-2):34-42), ANCA vasculitis (Savage. Clin. Exp. Immunol. Pathogenesis of anti-neutrophil cytoplasmic autoantibody (ANCA)-associated vasculitis. 2011. May; 164 Suppl 1:23-26), Goodpasture syndrome (Pedchenko et al. Curr. Opin. Nephrol. Hypertens. Goodpasture's Disease: molecular architecture of the autoantigen provides clues to etiology and pathogenesis. 2011. May; 20(3):290-296), anti-phospholipid syndrome (Muscal and Brey. Lupus. Antiphospholipid syndrome and the brain in pediatric and adult patients. April; 19(4):406-411).

The presence of auto-antibodies directed against Factor H has been reported mainly in the context of atypical hemolytic uremic syndrome (Dragon-Durey et al. J. Am. Soc. Nephrol. Clinical features of anti-factor H auto-antibody-associated hemolytic uremic syndrome. 2010. 21:2180-2187) and glomerulonephritis (Meri et al. J. Exp. Med. Activation of the alternative pathway of complement by monoclonal lambda light chains in membranoproliferative glomerulonephritis. 1992 Apr. 1; 175(4):939-50). Finally anti-factor H antibodies have been associated with early stage of non-small cell lung cancer (Amornsiripanitch et al. Clin. Cancer Res. Complement factor H autoantibodies are associated with early stage NSCLC. 2010. 16, 3226-3231).

Detection and/or quantification of allo-antibodies and auto-antibodies is/are medically relevant for diagnosis purposes, for monitoring the course of the disease, for evaluating the efficacy of a treatment.

As a third series of examples, numerous therapeutic antibodies have been authorized on the market since the mid 1980's. For example, muromonab-CD3 (Janssen-Cilag) was the first monoclonal antibody to be approved for human therapy as a potent immunosuppressant to reduce acute graft rejection. Their medical uses encompass cancer therapy, autoimmune diseases, viral or bacterial infection as well as neuro-degenerative diseases, to name a few.

One may understand that the quantification of circulating therapeutic antibodies in patients that were administered with such drugs enables a physician to monitor the blood levels of this drug, to correlate drug levels with a health benefit of the patient and to adjust the doses of therapeutic antibodies to be subsequently administered.

However, for medical purposes, the quantification of circulating antibodies shall be highly accurate and quantification accuracy will be met only in cases wherein relevant reference values are available for calibrating a quantification test. The availability of precise calibration reference values is particularly important when quantification of low level circulating antibodies, such as for example allo-antibodies and/or auto-antibodies.

Regarding quantification of circulating allo- or auto-antibodies, the in vitro use of human-derived antibodies for obtaining calibration reference values with the view of increasing accuracy of in vitro quantification assays is subject of infectious and ethical considerations and are from limited sources of production.

Furthermore, regarding quantification of circulating therapeutic antibodies, the in vitro use of these antibodies also for obtaining calibration reference values with the view of increasing accuracy of in vitro quantification assays is legally prohibited, since therapeutic antibodies are the subject of marketing authorizations having a scope restricted to the in vivo use in patients in need thereof.

Thus, for quantifying in vitro circulating target-specific therapeutic antibodies contained in a blood sample originating from an individual administered therewith, a target-specific calibration antibody, distinct from the target-specific therapeutic antibody to be quantified, is generally used. In routine Enzyme-Linked ImmunoSorbent Assays (or ELISAs), when these assays are used for detecting or quantifying antibodies of interest, detection of these antibodies of interest is usually performed by providing detectable secondary antibodies that bind to the Fc region of the antibody of interest to be tested.

However, for performing accurate calibrated ELISA assays, the provision of secondary antibodies having sufficiently similar binding properties to both the antibody used for calibration and to the antibody of interest to be tested is highly uncertain.

Altogether, the known methods for quantifying antibodies and calibration method thereof suffer from several drawbacks.

These drawbacks encompass a limited availability of low titre circulating antibodies when their use as both calibration antibodies and antibodies of interest is sought. The drawbacks also encompass a high number of false-positive and false-negative results especially when there is a lack of a relevant reference antibody standard.

According to the knowledge of the inventors, identification of these drawbacks of prior art assays for quantifying antibodies has not been disclosed yet, nor, by definition, technical means aimed at overcoming these drawbacks.

Hence, there is a need to provide standardized and accurate methods for quantifying antibodies.

There is also a need to provide standardized calibration antibodies, which can be obtained in large amounts, i.e. suitable for an industrial scale.

There is also a need to provide an uncoupling of the calibration method and a quantification method by quantifying distinct types of antibodies, i.e. the calibration antibody being distinct from the test antibody.

There is also a need to provide alternative means for detection of both the calibration antibody and the therapeutic antibody.

Finally, there is also a need to provide methods for the quantification of antibodies in a sample, which methods shall be specific, sensitive and reproducible.

### SUMMARY OF THE INVENTION

After many efforts to solve the technical problems of calibration, the inventors provided an in vitro method for quantifying a target-specific test antibody in a test sample.

Surprisingly, the inventors found that detectable non-antibody ligand that binds to the Fc region or to a light chain of an antibody may replace the conventionally used secondary antibody for both the calibration and the quantification assays.

More particularly, detectable non-antibody ligand that binds to the kappa light chains of an antibody is encompassed within the scope of the present invention.

Moreover, the inventors provide experimental evidences supporting the fact that calibration antibodies and test antibodies may be distinct antibodies, such as for example from distinct species.

Hence, the present invention relates to an in vitro method for quantifying a target-specific test antibody in a test sample, comprising the steps of:

a) performing an immunoassay using a target immobilized on a support which is brought into contact with the test sample, the immunoassay comprising a step of measuring the binding of the target-specific test antibody to the immobilized target by using a detectable non-antibody ligand that binds to the Fc region or to a light chain of an antibody, whereby a concentration-related value of the target-specific test antibody in the test sample is obtained, and

b) comparing the concentration-related value obtained at step a) with a reference value obtained by performing an immunoassay using the target immobilized on a support which is brought into contact with a calibration sample comprising a known concentration of a target-specific calibration antibody, the immunoassay comprising a step of measuring the binding of the target-specific calibration antibody to the immobilized target by using the detectable non-antibody ligand of step a),

and wherein:

(i) the target-specific test antibody of step a) and the target-specific calibration antibody of step b) are identical, or

(ii) the target-specific test antibody of step a) and the target-specific calibration antibody of step b) are distinct.

A further aspect of the present invention relates to a kit for quantifying a target-specific antibody in a test sample, comprising:
a target-specific calibration antibody, and;
a detectable non-antibody ligand that binds to the Fc region or to a light chain of an antibody.

### LEGEND TO THE FIGURES

FIG. 1 is a graph illustrating the validation of a method for quantifying anti-IgA antibodies according to the invention as compared to a routine method. Abscissa: quantification values as expressed in Arbitrary units (UA). Ordinate: quantification values as expressed as ng/ml of anti-IgA antibodies.

FIG. 2 is a representing graph illustrating the validation of a method for quantifying anti-factor H antibodies according to the invention as compared to a routine method. Abscissa: quantification values as expressed in Arbitrary units (UA). Ordinate: quantification values as expressed as ng/ml of anti-FH antibodies.

FIG. 3 is a graph illustrating the determination of the dose of eculizumab for efficient inhibition of plasma C5. Abscissa: total amount of eculizumab added (μg/ml) in plasma samples. Left ordinate: inhibition of plasma C5, expressed as CH50%, i.e. inhibition of 50% of C5 activity. Right ordinate: free eculizumab (μg/ml). Diamonds represent the CH50%. Squares represent free eculizumab, as measured by a classical ELISA method. Triangles represent free murine anti-C5 antibody (×10 μg/ml), as measured by the ELISA method according to the present invention.

### DETAILED DESCRIPTION OF THE INVENTION

### 1) Definitions

Antibody

The terms “antibody” and “immunoglobulin” are intended to be equivalent with respect to the present invention. An antibody according to the present invention may encompass any immunoglobulin (Ig), i.e. an immunoglobulin from any of the known classes from different species, such as the 5 classes of human immunoglobulins IgA, IgD, IgE, IgG and IgM immunoglobulins.

The antibodies according to the invention comprise heavy and light chains and possess a Fc region. Notably, the light chains of the antibodies according to the invention consist of kappa light chains.

The antibodies from the instant invention might be monoclonal antibodies, polyclonal antibodies, recombinant antibodies, chimeric antibodies, humanized antibodies and optimized antibodies, for example antibodies with modified glycosylation and antibodies having a variant Fc region having optimized binding affinity with one or more Fc receptors.

The antibodies from the instant invention might be monoclonal antibodies, polyclonal antibodies, recombinant antibodies, chimeric antibodies, humanized antibodies and optimized antibodies, having at least a light chain, which encompasses those having a kappa light chain.

Chimeric antibodies contain naturally occurring variable region (light chain and heavy chain) derived from an antibody from a given first species which is fused with the constant regions of the light chain and of the heavy chain derived from an antibody of a second species, distinct from the first species.

Antibodies suitable for the instant invention can be prepared using genetic recombination techniques. For example, a chimeric antibody can be prepared by cloning a DNA comprising a promoter and a sequence encoding the variable region of a non-human monoclonal antibody of the invention, including a murine monoclonal antibody of the invention, and the sequence encoding the constant region of another antibody, for example, the constant region of a murine antibody or other constant region of another human antibody. Such a chimeric antibody of the invention may for example be a mouse-mouse chimeric antibody or a chimeric mouse-human antibody, or every combination between 2 species.

Chimeric or humanized antibodies can be prepared using methods described by Jones et al. (Nature. 1986. Vol 321. 522-525) by Verhoeyen et al. (Science. 1988. Vol 239. 1534-1536) or by Riechmann et al. (Nature. 1988. Vol 322. 323-327). Chimeric or humanized antibodies can be prepared using techniques known to those skilled in the art such as those described by Singer et al. (J. Immun. 1992. Vol 150: 2844-2857), Mountain et al. (Biotechnol. Genet. Eng. Rev. 1992. Vol 10: 1-142) or Bebbington et al. (Biotechnology. 1992. Vol 10: 169-175).

Other techniques for antibody preparation by genetic recombination can be implemented according to the invention, which includes CDR grafting techniques are, for example those described in the documents by the following patents: EP 0451216, EP 0682040, EP 0939127, EP 0566647, U.S. Pat. No. 5,530,101, U.S. Pat. No. 6,054,297, U.S. Pat. No. 5,886,152 or U.S. Pat. No. 5,877,293.

Sample

Within the scope of the instant invention, the term “sample” is intended to encompass any biological fluid, cell, tissue, organ or portion thereof, including or potentially including a target-specific antibody, such an IgA, IgD, IgE, IgG or IgM. The term encompasses samples present in an individual as well as samples obtained or derived from the individual. For example, a sample can be a biological fluid, such as blood, serum, plasma, milk, lymph, and the like.

A sample also encompasses any material comprising a substance derived from any biological fluid, cell, tissue, organ or portion thereof, including or potentially including a target-specific antibody, such as an IgA, IgD, IgE, IgG or IgM from any species producing immunoglobulins. Thus, a sample encompasses liquid solutions comprising a substance derived from any biological fluid, cell, tissue, organ or portion thereof, including or potentially including a target-specific antibody, for example a blood or plasma or serum aliquot, which is diluted in a liquid solution such as a saline buffer.

Immunoassay

Immunoassays encompass any assay wherein a capture reagent is immobilized on a support and wherein detection of an analyte of interest is performed through the use of antibodies directed against the said analyte of interest.

As intended herein, immunoassays encompass those using a support selected in a group comprising beads (Luminex®, CBA®), a membrane (e.g. dot blot assays, Western blot assays, ELISPOT assays, etc), a plate (ELISA).

In the context of the present invention, a “capture reagent” is also termed “target” and an “analyte” and encompasses target-specific test antibodies and target-specific calibration antibodies.

The support used for immobilization of a capture reagent may be any inert support or carrier that is essentially water insoluble and useful in immunometric assays, including supports in the form of, e.g., surfaces, particles, porous matrices, etc. Examples of commonly used supports include small sheets, Sephadex, polyvinyl chloride, plastic beads, and assay plates or test tubes manufactured from polyethylene, polypropylene, polystyrene, and the like including 96-well microtiter plates, as well as particulate materials such as filter paper, agarose, cross-linked dextran, and other polysaccharides. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are suitably employed for capture reagent immobilization.

In a preferred embodiment the immobilized capture reagents are coated on a microtiter plate, and in particular the preferred solid phase used is a multi-well microtiter plate that can be used to analyze several samples at one time. Illustrations of multi-well microtiter plates encompass microtest 96-well ELISA plates such as that sold as Nunc Maxisorb® or Immulon®.

The capture reagent may be linked to the support by a non-covalent or covalent interaction or physical linkage as desired techniques for attachment include those described in U.S. Pat. No. 4,376,110 and the references cited therein. For performing a covalent linkage of the capture reagent to the support, the plate or other solid phase may be incubated with a cross-linking agent together with the capture reagent under conditions well known in the art such as for one hour at room temperature.

Commonly used cross-linking agents for attaching capture reagents to a solid support include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates capable of forming cross-links in the presence of light.

If plates are utilized (e.g., 96-well plates), they may be coated with a capture reagent using a variety of methods that are well known in the art.

The coated plates may then be treated with a blocking agent that binds non-specifically to and saturates the binding sites to prevent unwanted binding of an analyte of interest to the excess sites on the wells of the plate. Examples of appropriate blocking agents for this purpose include, for example, gelatin, bovine serum albumin, egg albumin, casein, and non-fat milk. Blocking treatment methods are well known by the one skilled in the art.

ELISA (Enzyme-Linked ImmunoSorbent Assay)

The term “ELISA” refers to a plate-based assay designed for detecting and quantifying substances such as peptides, proteins, lipids, nucleic acids, antibodies and hormones. In an ELISA, an antigen must be immobilized onto a support and then contacted with an antibody that is linked to a detectable mean, for example an enzyme or a fluorescent molecule. Detection is accomplished by assessing the conjugated enzyme activity via incubation with a substrate to produce a measureable product, or by assessing the fluorescence. The essential feature of the detection strategy is a highly specific antibody-antigen interaction.

This rather basic procedure may be performed with several modifications. First, the immobilization of the antigen of interest may be accomplished by direct adsorption to the assay plate (direct and indirect ELISA) or indirectly, for example via a capture antibody that has been attached to the plate (sandwich ELISA). Second, the antigen may be detected either directly (labelled primary antibody, direct ELISA) or indirectly (labelled molecule able to bind to the antibody bound to the antigen, indirect ELISA).

ELISA methods suitable for the instant invention are chosen among the indirect and the sandwich ELISA.

Any improved ELISA method may be suitable for performing the instant invention, such as, for example, the ELISA method described in U.S. Pat. No. 7,824,867. Notably, buffers allowing for quick coating and quick blocking may significantly reduce the time frame needed for performing the ELISA method.

Target

The term “target” encompasses any molecule containing an antigenic determinant (epitope) to which an antibody specifically binds, and hence is potentially able to provoke an immune response in a living organism bearing an immune system. According to the present invention “target” and “antigen” may be substituted to one another. Targets suitable for the present invention encompass but are not limited to nucleic acids, lipids, carbohydrates, proteins, glycoproteins, lipoproteins, peptides and the likes.

Immobilization on a Support

The present invention relies upon “a target immobilized on a support”. According to the protection sought in the present invention, “immobilization on a support” is referring to direct or indirect interactions that renders the target strongly associated to the support, and implies very stringent conditions to be removed. Interactions comprise covalent and affinity interactions. Affinity interactions are non-covalent interactions and comprise ionic, hydrogen, hydrophobic, Van Der Waals interactions. Hence, the target may be directly grafted onto a support, through for example covalent bonding. The target may be indirectly bound to the support, by the mean of (i) a spacer molecule covalently bound to the support and covalently bound to the target, (ii) an affinity molecule, such as a nucleic acid (DNA or RNA aptamer), an antibody or a fragment of an antibody, one extremity of the affinity molecule being bound to the support (covalently grafted) and the other extremity of the affinity molecule being bound to the target to be immobilized.

Bringing into Contact

Within the scope of the present invention “bringing into contact” refers to providing two compounds together, in conditions suitable for their interaction to take place. A skilled person in the art is capable of finding the suitable conditions, or the optimum conditions, with respect to reaction time, temperature, pH, buffer composition in order to promote these interactions. For example; the interactions between a target-specific antibody and a target form an antibody-antigen complex or conjugate.

Non-Antibody Ligand

A “ligand” is intended to refer to a molecule able to bind with high affinity to another molecule. Within the scope of the present invention a “non-antibody ligand” refers to a molecule, such as a protein, which does not consist of an antibody, the said molecule being able to bind specifically to the Fc region or to the light chain of an antibody.

Fc Region

The term “Fc region” refers to a C-terminal region of an immunoglobulin, in particular the C-terminal region of the heavy chain(s) of an immunoglobulin.

Light Chain

The light chain of an antibody consists in a constant domain and a variable domain. The variable domain is involved in the recognition of the epitope region of the target or antigen.

Kappa or Lambda Light Chain

The expression “kappa light chain” refers to one isotype of light chain, the second possible isotype being the “lambda light chain”. For example, in human, the ratio of kappa light chain over the lambda light chain is 2:1.

Reference Value

A “reference value” according to the present invention intends to relate to a numerical value representing the signal obtained from the detection of the binding of a non-antibody ligand to a known amount of a target-specific calibration antibody, following the implementation of the immunoassay described in the present invention.

Identical

The term “identical” intends to refer to a molecule from a defined species. Molecules from genotype polymorphism are encompassed within the scope of the present invention. Identical molecules must share the same biological properties. Hence, the term “identical” comprises isoforms of a same molecule, genetic variants of a same molecule from the same species. A skilled person in the art may consider that isoforms, genetic variants and the likes, sharing at least 85% identity based on a one to one alignment, are identical. It is understood that the identity value encompass 86%, 87%, 88%, 90%, 91%, 92%, 93%, 94%, 95%, 96%, 97%, 98%, 99% and 100%.

The comparison of the sequence optimal alignment may be performed by using known algorithms.

Most preferably, the sequence identity percentage is determined using the CLUSTAL W software (version 1.82) the parameters being set as follows: (1) CPU MODE=ClustalW mp; (2) ALIGNMENT=“full”; (3) OUTPUT FORMAT=“aln w/numbers”; (4) OUTPUT ORDER=“aligned”; (5) COLOR ALIGNMENT=“no”; (6) KTUP (word size)=“default”; (7) WINDOW LENGTH=“default”; (8) SCORE TYPE=“percent”; (9) TOPDIAG=“default”; (10) PAIRGAP=“default”; (11) PHYLOGENETIC TREE/TREE TYPE=“none”; (12) MATRIX=“default”; (13) GAP OPEN=“default”; (14) END GAPS=“default”; (15) GAP EXTENSION=“default”; (16) GAP DISTANCES=“default”; (17) TREE TYPE=“cladogram” and (18) TREE GRAP DISTANCES=“hide”.

Distinct

The term “distinct” intends to refer to a molecule from two different species, or molecules from the same species but different in their structure and/or function. A skilled person in the art may consider that isoforms, genetic variants and the like, from the same species, which are sharing less at least than 85% identity based on a one to one alignment, are distinct. It is understood that the identity value encompass less than 84%, 83%, 82%, 81%, 80%, 75%, 70%, 65%, 60%, 55%, 50%, 45%, 40%, 35%, 30%, 25%, 20%, 15%, 10%, 1%, 0.1%, 0.01%, 0.001%.

2) Method

A first aspect of the invention relates to an in vitro method for quantifying a target-specific test antibody in a test sample, comprising the steps of:

a) performing an immunoassay using a target immobilized on a support which is brought into contact with the test sample, the immunoassay comprising a step of measuring the binding of the target-specific test antibody to the immobilized target by using a detectable non-antibody ligand that binds to the Fc region or to a light chain of an antibody, whereby a concentration-related value of the target-specific test antibody in the test sample is obtained, and

b) comparing the concentration-related value obtained at step a) with a reference value obtained by performing an immunoassay using the target immobilized on a support which is brought into contact with a calibration sample comprising a known concentration of a target-specific calibration antibody, the immunoassay comprising a step of measuring the binding of the target-specific calibration antibody to the immobilized target by using the detectable non-antibody ligand of step a),

and wherein:

(i) the target-specific test antibody of step a) and the target-specific calibration antibody of step b) are identical, or

(ii) the target-specific test antibody of step a) and the target-specific calibration antibody of step b) are distinct.

In one preferred embodiment, a light chain of an antibody consists of a kappa light chain.

In preferred embodiments, the immunoassay is an ELISA.

In a preferred embodiment, the term “ELISA” encompasses an indirect ELISA, which comprises the following steps:

1) contacting a sample containing the target of interest to a solid support, in conditions suitable for immobilizing the target present in the sample to the solid support,

2) washing off the solid support so as to remove the unbound target molecules,

3) contacting a test sample susceptible to contain a target-specific test antibody able to bind to the immobilized target,

4) washing off the solid support,

5) contacting a detectable non-antibody ligand able to bind to the Fc region or to a light chain of an antibody, and

6) measuring the binding of the detectable non-antibody ligand to the Fc region or to the light chain of an antibody for determining the presence and amount of the target-specific test antibody in the test sample.

Indeed, steps 1) and 2) consist of the steps of preparing a ready-to-use ELISA assay format having a target-coated solid support, so that an ELISA assay generally starts at step 3) wherein a previously prepared assay format is used.

In another embodiment, the term “ELISA” also encompasses a sandwich ELISA, which comprises the following steps:

1) contacting a surface of a solid support with a target-specific antibody or a fragment thereof, and then washing off the support so as to remove the unbound target-specific antibody,

2) contacting a sample containing the target of interest to the solid support prepared in step 1) in conditions suitable for immobilizing the target present in the sample to the antibody or fragment thereof immobilized on the support,

3) washing off the solid support so as to remove the unbound target,

4) contacting a test sample susceptible to contain a target-specific test antibody able to bind the immobilized target,

5) washing off the solid support,

6) contacting a detectable non-antibody ligand able to bind to the Fc region or to a light chain of an antibody, and

7) measuring the binding of the detectable non-antibody ligand to the Fc region or to a light chain of an antibody for determining the presence and amount of the target-specific test antibody in the test sample.

An antibody or fragment thereof having target binding properties may also be suitable for immobilization on a support for the sandwich ELISA. By “antibody fragment” is meant a portion of an antibody such as Fab, Fab′, F(ab)₂, F(ab′)₂ fragments and the like. An “antibody fragment” also includes any synthetic or genetically engineered protein that can act as an antibody by binding to a detectable protein of the invention, in a protein complex as defined above.

An antibody or antibody fragment suitable for the invention may be prepared by any method known to those skilled in the art, as described, for example, in “Making and using antibodies: a practical handbook” (Howard & Kaser, Ed CRC, 2006).

In some embodiments, the target is immobilized onto a support by a spacer chain. The spacer chain may be of any known type and is intended to physically remove the target from the solid support surface on which said compound may be immobilized. Hence, a spacer chain provides a relative mobility of the target from the solid support surface on which it can be immobilized. The spacer chain further limits or prevents steric congestion due to the too close interaction of the solid support and that target, which interactions may interfere with binding of said target to the target-specific antibodies.

Advantageously, the spacer chain is linked to one end to the solid support and on the other end to the target. Preferably the spacer chain is a nonspecific or polyethylene glycol (PEG) or a hydrophilic hydrocarbon chain oligonucleotide. Suitable hydrophilic hydrocarbon chain oligonucleotides are DNA or RNA aptamers (often referred as to nucleic acid antibodies), which specifically bind to the target.

For either indirect or sandwich ELISA method, the calibration may be performed by using a target-specific calibration antibody in the place of the target-specific test antibody.

The test sample, which may comprise the target-specific test antibody, may be diluted with any suitable diluent. A skilled person in the art has the ability to select a suitable diluent from the bulk of described diluent. The diluent may be a buffered solution, such as, for example, a phosphate-buffered saline (PBS), a Tris Buffer Saline (TBS). The buffered solution may also be supplemented with saturating preparation such as Bovine Serum Albumin (BSA), low fat dry milk or gelatin, in order to limit non-specific interactions.

Dilution may range from 1:1 (v/v) to 1:60000 (v/v), with respect to the test sample over the diluent. This range encompasses 1:2, 1:3, 1:4, 1:5, 1:10, 1:20, 1:25, 1:50, 1:75, 1:100, 1:200, 1:250, 1:300, 1:400, 1:500, 1:600, 1:700, 1:800, 1:900, 1:1000, 1:2500, 1:5000, 1:7500, 1:10000, 1:20000, 1:25000, 1:30000, 1:40000, 1:50000, 1:60000 and intermediate values thereof.

In some embodiments, the test sample is diluted in the diluent in a ratio from 1:10 to 1:10000, advantageously in a ratio from 1:50 to 1:5000, more advantageously in a ratio from 1:50 to 1:2500.

In general, an “appropriate” contact time is the period of time that is sufficient to detect the presence of a specific-target test antibody within a test sample. Preferably, the contact time is sufficient to achieve a level of binding that is at least 95% of that achieved at equilibrium between bound and unbound target. A skilled in the art will recognize that the time necessary to achieve equilibrium may be readily determined by assaying the level of binding that occurs over a period of time (between 15 to 120 minutes). At room temperature, contacting the target with a target-specific antibody for a period of time of about 30 minutes is generally sufficient.

Suitable target-specific test antibody and/or target-specific calibration antibody for the present invention encompass(es) chimeric or humanized antibody/antibodies, including antibodies from different mammal species and that are recognized by the same non-antibody ligand.

For example, the target-specific test antibody may be human and the target-specific calibration antibody may be a chimeric antibody, or alternatively, the target-specific test antibody may be a chimeric antibody and the target-specific calibration antibody may be human.

Chimeric antibodies encompass antibodies having a fragment belonging to a species and another fragment belonging to another species. For example; a chimeric antibody suitable for the invention may comprise a F(ab)₂ region from human and a Fc region from mouse, F(ab)₂ region from rat and a Fc region from mouse, F(ab)₂ region from goat and a Fc region from human, etc.

In other embodiments, the target-specific test antibody may be a human antibody and the target-specific calibration antibody may be a humanized antibody, or alternatively, the target-specific test antibody may be a humanized antibody and the target-specific calibration antibody may be a human antibody.

In still further embodiments, the target-specific test antibody may be a human antibody and the target-specific calibration antibody may be a humanized antibody, or alternatively, the target-specific test antibody may be a humanized antibody and the target-specific calibration antibody may be a human antibody.

In yet further embodiments, both the target-specific test antibody and the target-specific calibration antibody may be chimeric antibodies.

In still other embodiments, the target-specific test antibody may be a chimeric antibody and the target-specific calibration antibody may be a humanized antibody, or alternatively, the target-specific test antibody may be a humanized antibody and the target-specific calibration antibody may be a chimeric antibody.

In yet other embodiments, both the target-specific test antibody and the target-specific calibration antibody may be humanized antibodies.

In preferred embodiments of the in vitro method according to the invention, (i) the target-specific test antibody of step a) and the target-specific calibration antibody of step b) originate from the same animal species, or alternatively (ii) the target-specific test antibody of step a) and the target-specific calibration antibody of step b) originate from distinct animal species.

For example, both the target-specific test antibody and the target-specific calibration antibody are human antibodies.

According to the instant invention, human antibodies encompass IgA, IgD, IgE, IgG1, IgG2, IgG3, IgG4 and IgM antibodies.

In further embodiments of the in vitro method according to the invention (i) the target-specific test antibody and (ii) the target-specific calibration antibody is independently selected in a group comprising human and non-human antibodies.

Advantageously, the non-human antibody which may be used in the in vitro method as described herein is selected in a group comprising mammal antibodies, avian antibodies, amphibian antibodies, reptile antibodies, fish antibodies and insect antibodies.

In some embodiments, the non-human antibody is selected from antibodies from non-human animal of economic interest. Non-human animal of economic interest may be selected in a group comprising cat, cattle, dog, goat, goose, guinea pig, hamster, horse, lama, monkey, mouse, pig, poultry, rabbit, rat, sheep, salmon, swine.

In other embodiments of the in vitro method according to the present invention, the target-specific test antibody is a human antibody.

In a still other embodiments, the in vitro method according to the present invention is implemented with a target-specific test antibody selected in a group comprising auto-antibodies, allo-antibodies, therapeutic antibodies and imaging antibodies.

Auto-antibodies that may be detected or quantified by an in vitro method as described herein may be selected in a group comprising (i) antinuclear antibodies, comprising anti-SSA/Ro auto-antibodies, anti-La/SS-B auto-antibodies, anti-centromere antibodies, anti-neuronal nuclear antibody-2, anti-dsDNA, anti-RNP, anti-Smith, anti-topoisomerase antibodies, anti-histone antibodies, anti-p62 antibodies, anti-sp100 antibodies; (ii) anti-glycoprotein 210 antibodies; (iii) anti-transglutaminase antibodies, comprising anti-tTG antibodies and anti-eTG antibodies; (iv) anti-ganglioside antibodies; (v) anti-actin antibodies; (vi) anti-CCP; liver kidney microsomal type 1 antibody; (vii) anti-thrombin antibodies; (viii) anti-neutrophil cytoplasmic antibody (ANCA) comprising anti-myéloperoxydase (MPO), anti-proteinase 3 (PR3), anti-lactoferrine, anti-elastase, anti-bacterial inducing protein (BPI), anti-cathepsine G, (ix) anti-glomerular basement membrane (alpha 3 chain of Collagen 4), anti-phospholipase A2 receptor (PLA2R); (x) anti-rheumatoid factor antibodies; (xi) anti-smooth muscle antibody, comprising anti-actin antibodies, anti-troponin antibodies and anti-tropomyosin antibodies; (xii) anti-mitochondrial antibodies, comprising anti-cardiolipin antibodies, anti-sulfite oxidase antibodies, anti-sarcosine dehydrogenase antibodies and anti-glycogen phosphorylase antibodies; (xiii) anti-SRP antibodies; anti-VGCC (voltage-gated calcium channel) antibodies; (xiv) anti-VGKC (voltage-gated potassium channel) antibodies; (xv) anti-synthetase antibodies comprising anti-PL7, -PL12, -JO1, -EJ, -OJ antibodies and (xvi) anti-complement pathway antibodies, comprising anti-factor H auto-antibodies, anti-C1 Inhibitor, anti-C1q, anti-C3, anti-Factor B, anti-C3bBb (C3 convertase of the complement alternative pathway), anti-C4b2a (C3 convertase of the complement classical pathway).

In a preferred embodiment, the target-specific test antibody is an auto-antibody, preferably an anti-factor H auto-antibody, anti-C1 Inhibitor, anti-C1q, anti-C3, anti-Factor B.

Allo-antibodies that may be detected or quantified by an in vitro method as described herein may be selected in a group comprising anti-human platelet antigens (HPA) antibodies, anti-IgA antibodies.

In a preferred embodiment, the target-specific test antibody is an allo-antibody, preferably an anti-IgA antibody.

Human therapeutic antibodies that may be detected or quantified by an in vitro method as described herein may be selected in a group comprising Panitumumab. Actoxumab, Adalimumab, Adecatumumab, Alirocumab, Anifrolumab, Atinumab, Atorolimumab, Belimumab, Bertilimumab, Bezlotoxumab, Bimagrumab, Briakinumab, Brodalumab, Canakinumab, Carlumab, Cixutumumab, Conatumumab, Daratumumab, Denosumab, Drozitumab, Duligotumab, Dupilumab, Dusigitumab, Efungumab, Eldelumab, Enoticumab, Evolocumab, Exbivirumab, Fasinumab, Fezakinumab, Figitumumab, Flanvotumab, Foralumab, Foravirumab, Fresolimumab, Fulranumab, Ganitumab, Gantenerumab, Glembatumumab vedotin, Golimumab, Guselkumab, Icrucumab, Inclacumab, Intetumumab, Ipilimumab, Iratumumab, Lerdelimumab, Lexatumumab, Libivirumab, Lirilumab, Lucatumumab, Mapatumumab, Mavrilimumab, Metelimumab, Morolimumab, Namilumab, Narnatumab, Nebacumab, Necitumumab, Nesvacumab, Nivolumab, Ofatumumab, Olaratumab, Orticumab, Oxelumab, Panitumumab, Panobacumab, Parsatuzumab, Patritumab, Placulumab, Pritumumab, Radretumab, Rafivirumab, Ramucirumab, Raxibacumab, Regavirumab, Rilotumumab, Robatumumab, Roledumab, Sarilumab, Secukinumab, Seribantumab, Sevirumab, Sirukumab, Stamulumab, Tabalumab, Teprotumumab, Ticilimumab (=tremelimumab), Tovetumab, Tralokinumab, Tremelimumab, Tuvirumab, Urelumab, Ustekinumab, Vantictumab, Vesencumab, Votumumab, Zalutumumab, Zanolimumab, Ziralimumab.

Murine therapeutic antibodies that may be detected or quantified by an in vitro method as described herein may be selected in a group comprising Abagovomab, Afelimomab, Anatumomab mafenatox, Blinatumomab, Detumomab, Dorlimomab aritox, Edobacomab, Edrecolomab, Elsilimomab, Enlimomab pegol, Epitumomab cituxetan, Faralimomab, Gavilimomab, Ibritumomab tiuxetan, Imciromab, Inolimomab, Lemalesomab, Maslimomab, Minretumomab, Mitumomab, Moxetumomab pasudotox, Muromonab-CD3, Nacolomab tafenatox, Naptumomab estafenatox, Nerelimomab, Odulimomab, Oregovomab, Pemtumomab, Racotumomab, Solitomab, Taplitumomab paptox, Telimomab aritox, Tenatumomab, Tositumomab, Vepalimomab and Zolimomab aritox.

Chimeric therapeutic antibodies that may be detected or quantified by an in vitro method as described herein may be selected in a group comprising Abciximab, Amatuximab, Basiliximab, Bavituximab, Brentuximab vedotin, Cetuximab, Clenoliximab, Ecromeximab, Ensituximab, Futuximab, Galiximab, Girentuximab, Gomiliximab, Indatuximab ravtansine, Infliximab, Keliximab, Lumiliximab, Pagibaximab, Priliximab, Pritoxaximab, Rituximab, Setoxaximab, Siltuximab, Teneliximab, Ublituximab, Vapaliximab, Volociximab and Zatuximab.

Humanized therapeutic antibodies that may be detected or quantified by an in vitro method as described herein may be selected in a group comprising Afutuzumab, Alacizumab pegol, Alemtuzumab, Anrukinzumab, Apolizumab, Aselizumab, Atlizumab (=tocilizumab), Bapineuzumab, Benralizumab, Bevacizumab, Bivatuzumab mertansine, Blosozumab, Cantuzumab mertansine, Cantuzumab ravtansine, Caplacizumab, Cedelizumab, Certolizumab pegol, Citatuzumab bogatox, Clazakizumab, Clivatuzumab tetraxetan, Concizumab, Crenezumab, Dacetuzumab, Daclizumab, Dalotuzumab, Demcizumab, Eculizumab, Efalizumab, Elotuzumab, Enavatuzumab, Enokizumab, Epratuzumab, Erlizumab, Etaracizumab, Etrolizumab, Farletuzumab, Felvizumab, Ficlatuzumab, Fontolizumab, Gemtuzumab ozogamicin, Gevokizumab, Ibalizumab, Imgatuzumab, Inotuzumab ozogamicin, Itolizumab, Ixekizumab, Labetuzumab, Lambrolizumab, Lampalizumab, Lebrikizumab, Ligelizumab, Lintuzumab, Lodelcizumab, Lorvotuzumab mertansine, Margetuximab, Matuzumab, Mepolizumab, Milatuzumab, Mogamulizumab, Motavizumab, Natalizumab, Nimotuzumab, Ocaratuzumab, Ocrelizumab, Olokizumab, Omalizumab, Onartuzumab, Oportuzumab monatox, Ozanezumab, Ozoralizumab, Palivizumab, Pascolizumab, Pateclizumab, Perakizumab, Pertuzumab, Pexelizumab, Pidilizumab, Pinatuzumab vedotin, Polatuzumab vedotin, Ponezumab, Quilizumab, Ranibizumab, Reslizumab, Romosozumab, Rontalizumab, Rovelizumab, Ruplizumab, Samalizumab, Sibrotuzumab, Sifalimumab, Simtuzumab, Siplizumab, Solanezumab, Sonepcizumab, Sontuzumab, Suvizumab, Tacatuzumab tetraxetan, Tadocizumab, Talizumab, Tanezumab, Tefibazumab, Teplizumab, Tildrakizumab, Tigatuzumab, Tocilizumab (=atlizumab), Toralizumab, Trastuzumab, Tregalizumab, Tucotuzumab celmoleukin, Urtoxazumab, Vatelizumab, Vedolizumab, Veltuzumab, Visilizumab and Vorsetuzumab mafodotin.

Bispecific therapeutics antibodies are artificial antibodies that are composed of fragments from two different antibodies and consequently have the capacity of binding to two different types of antigen.

Bispecific therapeutics antibodies that may be detected or quantified by an in vitro method as described herein may be selected in a group comprising Blinatumomab (Klinger et al. Blood. Immuno-pharmacologic response of patients with B-lineage acute lymphoblastic leukemia to continuous infusion of T cell-engaging CD19/CD3-bispecific BiTE antibody blinatumomab. 2012 Jun. 28; 119(26):6226-33; Topp et al. Blood. Long-term follow-up of hematologic relapse-free survival in a phase 2 study of blinatumomab in patients with MRD in B-lineage ALL. 2012 Dec. 20; 120(26):5185-7), anti-CEA/anti-diethylenetriaminepentaaceticacid (DTPA) bispecific antibody (Salaun. J. Nucl. Med. Phase II trial of anti-carcino-embryonic antigen pre-targeted radio-immunotherapy in progressive metastatic medullary thyroid carcinoma: biomarker response and survival improvement. 2012 August; 53(8):1185-92).

Therapeutic antibodies may also be conjugated with chimiotherapy agent.

Therapeutic antibodies conjugated with chimiotherapy agent that may be detected or quantified by an in vitro method as described herein may be selected in a group comprising Trastzumab-Emtansine, Brentuximab-Vedotin.

Imaging antibodies provide sensitive, non-invasive means for molecular characterization of cell surface phenotype in vivo, and hence may be useful for diagnosis, prognosis, therapy selection, and monitoring of treatment of diseases.

In a preferred embodiment, the target-specific test antibody is a therapeutic antibody, preferably the Eculizumab therapeutic antibody.

Although the following list is not intended to be limitative, the therapeutic antibodies that may be detected or quantified by an in vitro method as described herein may be useful in treating a disease selected in a group comprising acute myelogenous leukaemia; adrenocortical carcinoma; allergic asthma; Alzheimer's disease; ankylosing spondylitis; anthrax intoxication; arthritis; asthma; atopic diseases; autoimmune diseases; B-cell cancers; B-cell lymphoma; bleeding; brain cancer; breast cancer; choroidal and retinal neovascularization; chronic asthma; chronic hepatitis B; chronic lymphocytic leukaemia; clear cell renal cell carcinoma; Clostridium difficile infection; colorectal cancer; Crohn's disease; cytomegalovirus infection; dermatomyositis; diabetes mellitus type 1; diarrhoea caused by E. coli; focal segmental glomerulosclerosis; follicular lymphoma; graft versus host disease; haemorrhagic shock; head cancer; heart attack; hematologic cancers; haemolytic disease of the new-born; hepatitis B; HIV infection; Hodgkin's lymphoma; hypercholesterolemia; hypocholesterolemia; idiopathic pulmonary fibrosis; immunologically mediated inflammatory disorders; infectious disease/influenza A; inflammations of the airways, skin and gastrointestinal tract; inflammatory bowel disease; invasive Candida infection; juvenile idiopathic arthritis; lung cancer; lupus erythematosus; lupus nephritis nasopharyngeal cancer; lymphoma; macular degeneration (wet form); malignant melanoma; metastatic cancer; metastatic colorectal cancer; multiple myeloma; multiple sclerosis; muscular dystrophy; neuroblastoma; neck cancer; non-Hodgkin lymphoma; non-small cell lung carcinoma; organ transplant rejections; osteoporosis; ovarian cancer; pancreatic cancer; paroxysmal nocturnal haemoglobinuria; polymyositis; prostate cancer; psoriasis; psoriatic arthritis; Pseudomonas aeruginosa infection; rheumatic diseases; rheumatoid arthritis; sepsis; severe allergic disorders; small cell lung carcinoma; solid tumors; squamous cell carcinoma; Staphylococcus aureus infection; stomach cancer; stroke; systemic lupus erythematosus; systemic scleroderma; T-cell lymphoma; traumatic shock; ulcerative colitis; uveitis; viral infections; white blood cell diseases hemolytic uremic syndrome (HUS), membranoproliferative glomerulonephritis (MPGN) comprising dense deposit disease (DDD), C3 glomerulopathies.

In a most preferred embodiment of the in vitro method which is described herein, the target-specific test antibody that may be detected or quantified is a human antibody and the target-specific calibration antibody is a non-human antibody, preferably selected in a group comprising mouse antibodies, rat antibodies, llama antibodies, goat antibodies, sheep antibodies, rabbit antibodies and horse antibodies, and is preferably mouse antibodies.

In another preferred embodiment both the target-specific test antibody and the target-specific calibration antibody are non-human antibodies.

Advantageously, the detectable non-antibody ligand within the scope of the instant invention may be selected in a group comprising protein A, protein G, protein A/G, protein L and is preferably protein G.

To the knowledge of the inventors, proteins A, G and A/G have been widely used for antibodies purification. They were also used, for antibody detection (Dahlbom et al. Clin. Chim. Acta. 2008. Protein A and protein G ELISA for the detection of IgG autoantibodies against tissue transglutaminase in childhood celiac disease. September; 395(1-2):72-6) but were not reported to be useful for quantification using an antibody for the calibration from another species as described herein.

Protein A is a 56 kDa surface protein originally found in the cell wall of the bacterium Staphylococcus aureus. Native protein A presents 5 domains able to bind to a Fc region from several immunoglobulins.

Protein G is an immunoglobulin-binding protein expressed in Streptococcal bacteria from group C (58 kDa, namely C40 protein G) and from group G (65-kDa, namely G148 protein G). Natural protein G presents 2 domains able to bind to a Fc region from several immunoglobulins.

According to the instant invention, protein A and/or protein G may be naturally occurring purified proteins, or purified recombinant proteins. Preferably, recombinant protein A and/or protein G present(s) at least one Fc region binding domain. Preferably recombinant protein A presents at least 2 Fc region binding domains, preferably 3 Fc region binding domains, and preferably 4 Fc region binding domains.

Protein A/G is a recombinant fusion protein that combines the Fc region binding domains of both protein A and protein G. Protein A/G contains four Fc binding domains from protein A and two from protein G.

A skilled person in the art has the common knowledge to determine which protein from protein A, protein G and protein A/G may be the most suitable as a non-antibody ligand to bind the Fc region bearing target-specific calibration antibodies and/or the Fc region bearing target-specific test antibodies of interest. Indeed, it is commonly admitted that protein A and protein G are not able to bind any Fc region from any antibodies.

In another preferred embodiment, protein L may be used as the detectable non-antibody ligand.

Protein L is a 719 amino acid residues protein, which is present in the cell wall of Peptostreptoccus magnus. Protein L binds antibodies through interactions with the light chains. Hence, Protein L binds to single chain variable fragments (scFv) and Fab fragments. Protein L is disclosed notably by Murphy et al. (Amplified expression and large-scale purification of protein L. Bioseparation. 1996. 6(2):107-1).

Mechanistically, protein L binding is restricted to those antibodies that contain kappa light chains. However, protein L is only effective in binding certain subtypes of kappa light chains. For example, protein L binds to human VκI, VκIII and VκIV subtypes of kappa light chains but does not bind the VκII subtype of kappa light chains.

Within the scope of the invention, it is important to understand that binding of protein L to a kappa light chain of an antibody does not interfere with the binding of said antibody to its target. Indeed, binding of protein L to a kappa light chain of an antibody does not involve the hypervariable regions of the antibody, which are taking part in the binding with the target.

Table 1 below describes the binding affinities of protein A, protein G and protein L, towards commonly used antibodies.

| | Nature of the Fc | | | |
|---|---|---|---|---|
| | region bearing | Protein A | Protein G | Protein L |
| Organism | immunoglobulin | affinity | affinity | affinity |
| Human | IgG1 | + | + | + |
| | IgG2 | + | + | + |
| | IgG3 | − | + | + |
| | IgG4 | + | + | + |
| | IgA | + | − | + |
| | IgD | + | − | + |
| | IgE | + | − | + |
| | IgM | + | − | + |
| Mouse | IgG1 | + | + | + |
| | IgG2a | + | + | + |
| | IgG2b | + | + | + |
| | IgG3 | + | + | + |
| | IgM | + | − | + |
| Rat | IgG1 | − | + | + |
| | IgG2a | − | + | + |
| | IgG2b | − | + | + |
| | IgG2c | + | + | + |
| | IgM | + | − | ? |
| Rabbit | Total Ig | + | + | + |
| Hamster | Total Ig | + | + | + |
| Guinea Pig | Total Ig | + | + | ? |
| Bovine | Total Ig | + | + | − |
| Sheep | Total Ig | + | + | − |
| Goat | Total Ig | + | + | − |
| Pig | Total Ig | + | + | + |
| Chicken | Total Ig | − | + | − |

? stands for “unknown binding affinity”.

Table 2 below describes the relative binding affinities of protein A, protein G, protein A/G and protein L, towards a subset of commonly used antibodies.

| | Protein A | Protein G | Protein A/G | Protein L |
|---|---|---|---|---|
| Human IgG | S | S | S | S |
| Human IgG1 | S | S | S | S |
| Human IgG2 | S | S | S | S |
| Human IgG3 | w | S | S | S |
| Human IgG4 | S | S | S | S |
| Human IgM | w | nb | w | S |
| Human IgA | w | nb | w | S |
| Human IgA1 | w | nb | S | S |
| Human IgA2 | w | nb | S | S |
| Human IgD | nb | nb | w | S |
| Mouse IgG | S | S | S | S |
| Mouse IgG1 | w | w/s | w/s | S |
| Mouse IgG2a | S | S | S | S |
| Mouse IgG2b | S | S | S | S |
| Mouse IgG3 | S | S | S | S |
| Mouse IgM | nb | nb | nb | S |
| Horse IgG | w | S | S | nt |
| Horse IgG(c) | w | nb | w | nt |
| Horse IgG(T) | nb | nb | nb | nt |
| Rabbit IgG | S | S | S | w |
| Goat IgG | w | S | S | nb |
| Rat IgG | w | w/s | w/s | S |
| Sheep IgG | w | S | S | nb |
| Cow IgG | w | S | S | nb |
| Guinea Pig IgG | S | w | S | nt |
| Pig IgG | S | w | S | S |
| Dog IgG | S | w | S | nt |
| Cat IgG | S | w | S | nt |
| Monkey IgG (Rhesus) | S | S | S | nt |
| Chicken IgG | nb | nb | nb | nb |

w stands for a “weak binding”; S stands for a “strong binding”; w/s stands for “indifferent binding”; nb stands for “no binding”; nt stands for “not tested”.

In some embodiments, when the Fc region of the target-specific test antibodies and/or target-specific calibration antibodies are from human IgG1, IgG2 and IgG4 immunoglobulins, the detectable non-antibody ligand is selected in a group comprising protein A, protein G and protein A/G, preferably protein G.

In some embodiments, when the Fc region of the target-specific test antibodies and/or target-specific calibration antibodies are from human IgG3 immunoglobulins, the detectable non-antibody ligand is selected in a group comprising protein G and protein A/G, preferably protein G.

In some embodiments, when the Fc region of the target-specific test antibodies and/or target-specific calibration antibodies are from human IgA, IgD, IgE and IgM immunoglobulins, the detectable non-antibody ligand is selected in a group comprising protein A and protein A/G.

In some embodiments, when the Fc region of the target-specific test antibodies and/or target-specific calibration antibodies are from mouse IgG1, IgG2a, IgG2b and IgG3 immunoglobulins, the detectable non-antibody ligand is selected in a group comprising protein A, protein G and protein A/G, preferably protein G.

In some embodiments, when the Fc region of the target-specific test antibodies and/or target-specific calibration antibodies are from mouse IgM immunoglobulins, the detectable non-antibody ligand is selected in a group comprising protein A and protein A/G.

In some embodiments, when the Fc region of the target-specific test antibodies and/or target-specific calibration antibodies are from rat IgG1, IgG2a and IgG2b immunoglobulins, the detectable non-antibody ligand is selected in a group comprising protein G and protein A/G, preferably protein G.

In some embodiments, when the Fc region of the target-specific test antibodies and/or target-specific calibration antibodies are from rat IgG2c immunoglobulins, the detectable non-antibody ligand is selected in a group comprising protein A, protein G and protein A/G, preferably protein G.

In some embodiments, when the Fc region of the target-specific test antibodies and/or target-specific calibration antibodies are from rat IgM immunoglobulins, the detectable non-antibody ligand is selected in a group comprising protein A and protein A/G.

In some embodiments, when the Fc region of the target-specific test antibodies and/or target-specific calibration antibodies are from rabbit, hamster, guinea pig, bovine, sheep, goat, cats, dogs, horses and pig immunoglobulins, the detectable non-antibody ligand is selected in a group comprising protein A, protein G and protein A/G, preferably protein G.

In some embodiments, when the Fc region of the target-specific test antibodies and/or target-specific calibration antibodies are from chicken immunoglobulins, the detectable non-antibody ligand is selected in a group comprising protein G and protein A/G, preferably protein G.

In some embodiments, when the light chains, in particular the kappa light chain, of the target-specific test antibodies and/or target-specific calibration antibodies are from human, mouse, rat or pig immunoglobulins, the detectable non-antibody ligand may be protein L.

In a preferred embodiment, the detectable non-antibody ligand is labelled with a detectable molecule.

As for example of a detectable molecule, suitable to be used in the present invention, a skilled person in the art may refer to the non-limiting following list:
a radio-labelled molecule, in particular, a radioactive moiety suitable for the invention may for example be selected within the group comprising ³H, ¹²¹I, ¹²³I, ^{99m}Tc, ¹⁴C or ³²P;
a chemo-luminescent molecule (chromophore-labelled) or a fluorophore-labelled molecule, wherein a luminescent marker, and in particular a fluorescent marker, suitable for the invention may be any marker commonly used in the field such as fluorescein, BODIPY, fluorescent probes type ALEXA, coumarin and its derivatives, phycoerythrin and its derivatives, or fluorescent proteins such as GFP or the DsRed;
a polymer-backbone-molecule,
an enzyme-labelled molecule, said labelling enzyme suitable for the invention may be an alkaline phosphatase, a tyrosinase, a peroxydase, or a glucosidase; for example, suitable avidin-labelled enzyme may be an avidin-Horse Radish Peroxydase (HRP), and a suitable substrate may be AEC, 5-bromo-4-chloro-3-indolyl phosphate (BCIP), nitro blue tetrazolium chloride (NBT);
a molecule conjugated with a substrate or with the protein or ligand of a protein-ligand pair, in particular a biotin, a streptavidin;

In a preferred embodiment of the invention, the detectable molecule is selected in a group comprising a radioactive molecule, a chemo-luminescent molecule, a fluorescent molecule, a fluorophore and an enzyme.

In a still preferred embodiment, the in vitro method according to the invention is implemented with a test sample, which is selected in a group comprising a blood sample, a plasma sample, a serum sample, a lymph sample, a cerebrospinal fluid sample, an urine sample and a milk sample.

Blood, lymph; cerebrospinal fluid, urine and milk may be collected from an individual.

Starting from a whole blood sample, plasma and serum fractions may be obtained by classical methods known from a skilled in the art.

In some embodiments, the test sample according to the instant invention may be frozen and unfrozen and/or lyophilized before use. When a test sample is a dry powder, obtained by lyophilisation, the test sample may be suspended as a liquid solution with a suitable diluent. For example, lyophilized plasma may be suspended with sterile water before use.

In a preferred embodiment, the target-specific calibration antibody may be purified from a biological fluid sample comprising blood, plasma, serum, lymph, cerebrospinal fluid, urine, milk, ascite.

3) Kits

A further aspect of the invention relates to a kit for quantifying a target-specific antibody in a test sample, comprising:
a target-specific calibration antibody, and;
a detectable non-antibody ligand that binds to the Fc region or to a light chain of an antibody.

In a preferred embodiment, the kit according to the invention further comprises the target.

In some embodiments, the target may be under the form of a dry powder, obtained by lyophilisation. The powder is suspended in a liquid solution to obtain a target sample solution, which may be further diluted with a suitable diluent.

In some embodiments, the target is in liquid form, ready to be used as such or diluted with a suitable diluent.

In another preferred embodiment, the kit according to the instant invention further comprises one or more reagents for detecting the non-antibody ligand.

These reagents are not limited to buffers, for example a wash buffer, a diluent buffer, a stop buffer; colorimetric substrates and the like.

In a preferred embodiment, the kit according to the present invention also comprises instructions or a protocol indicating how to perform the assay.

Advantageously, the detectable non-antibody ligand is selected in a group comprising protein A, protein G, protein A/G and protein L.

In some embodiments, the kit according to the present invention comprises a support which may be pre-coated with the target, pre-coated with an antibody able to bind the target or pre-coated with an antibody on which is bound the target. Supports encompass microtiter plates, beads, filter membranes, gels, such as, for example, agarose gel, acrylamide gel, etc.

In preferred embodiments, the kit according to the present invention comprises a multiple well microplate, which may be pre-coated with the target, pre-coated with an antibody able to bind the target or pre-coated with an antibody on which is bound the target.

A solid support suitable for the ELISA may be a 96, 384 or 1536 well microplate, made of polystyrene, polypropylene or cyclo-olefin.

In a preferred embodiment, a kit according to the present invention may be used to quantify circulating naturally occurring antibodies, circulating antibodies associated with a medical condition (disease), circulating antibodies after a graft, circulating therapeutic antibodies.

In some embodiments, the kit according to the instant invention may be useful to quantify the efficacy of the administration of a therapeutic antibody. The quantification may be useful to adapt the treatment, by administrating the suitable dosage to an individual in need thereof.

In another embodiment, the kit according to the instant invention may be useful to detect antibodies for diagnosis purposes, such as for example diagnosing allo-antibodies or auto-antibodies.

In another embodiment, the kit according to the instant invention may be useful to detect antibodies for surveying the outcome of a treatment against an auto-antibody or an allo-antibody related disease. Administration of a suitable drug may reduce the circulating levels of, or deplete the individual of, the auto-antibody or the allo-antibody.

### EXAMPLES

### 1) Example 1

### Quantification of Serum Anti IgA Allo-Antibodies Concentration

Anti-IgA alloantibodies are developed in patients presenting with IgA deficiency. These patients will be allo-immunized during administrated by blood-derived products, such as fresh frozen plasma, intravenous Ig, etc. Selective IgA deficiency is the most frequent primary immunodeficiency in Europe and North America, with a prevalence estimated at 1/600. Because most subjects with selective IgA deficiency are asymptomatic, searching for anti-IgA antibodies is highly recommended for patients who have had adverse reactions or intolerance reactions during administration of blood products.

The assay currently used in routine is an ELISA, which assay requires the use of a human standard coming from a patient serum sample. This use induces problems about conservation, stock depletion and ethics.

1.1) Materials and Methods

a) Quantification Method According to the Invention

Purified human monoclonal IgA kappa (Cappel) is coated in wells of a microplate (50 μl of a solution at a concentration of 10 μg/ml per well) over night at a temperature of 4° C. Excess of unbound purified human polyclonal IgA kappa are removed from the wells. After a saturation step for 1 hour, at room temperature with 200 μl per well of PBS buffer containing 0.1% Tween 20, the wells are thoroughly washed with the same buffer. Individual test samples, namely serum samples, comprising IgA-specific antibodies, are diluted 1:100 in PBS containing 0.1% Tween 20 and are assayed according to a direct ELISA method.

Calibration of the ELISA was operated with a murine anti-IgA monoclonal antibody (anti-human IgA, clone AD3, ABCAM), at 7 concentrations, the monoclonal antibody was first diluted at 1:800 then a serial dilution 1:2 is performed until the dilution 1:51200.

After incubation for one hour at room temperature and washes with 200 μl/well of PBS, 0.1% Tween 20, a solution of G protein conjugated to peroxidase (Hpr-protein G from GenScript, catalog product number M00090) is applied (diluted at 1:6000 in PBS, 0.1% Tween 20, i.e. at a concentration of 167 ng/ml). After washes, a chromogenic enzyme substrate, the O-phenylenediamine (OPD, Sigma Aldrich) is mixed. The resulting peroxidation reaction provides a coloring of the solution, which can be accurately measured at 490 nm by spectrophotometry (with the reader Dynex-Technologies, using the software Revelation MRX, ThermoScientific). This method allows for the quantification of the allo-antibodies anti-IgA captured in the wells.

The validation procedure of the test was based on the recommendations from the COFRAC (GTA SH 04).

The evaluation of the limit of detection and limit of quantification, linearity, as well as the determination of biological reference interval, repeatability, intermediate precision, accuracy and intra-laboratory reproducibility were examined.

Notably, the method is statistically relevant when coefficients of variation and biases are below 20%.

b) Quantification Method According to a Reference Method

We also studied the correlation with the method according to the invention with a reference method routinely used in the laboratory.

Calibration of the ELISA assay was performed with a human serum sample containing anti-IgA allo antibodies, at 4 concentrations (the serum is diluted first at 1:300 then a serial dilution 1:2 is performed until the dilution 1:2400).

After incubation for one hour at room temperature, and washes with 200 μl/well of PBS, 0.1% Tween 20, a solution of a murine monoclonal anti-human IgG conjugated with HRP, diluted at 1:500 in PBS, 0.1% Tween 20, is added. After washes, the OPD enzyme substrate is mixed. Detection of the resulting peroxidation reaction is performed as above.

c) Assay Comparing the Method of Quantification According to the Invention and a Reference Method

30 samples containing varying known concentrations of IgG anti-IgA (50-3365 ng/ml) were processed accordingly to the 2 quantification methods described above.

1.2) Results

The measuring range was between 50 and 500 ng/mL as defined by the limit of quantification and the maximum of the reference curve. Lower limit of detection was 15 ng/ml. Samples were 1:100 diluted. If necessary, additional dilutions were performed. We evaluated repeatability of 3 control levels (High=2316 ng/ml, mean=1039 ng/ml and low=283 ng/ml). The coefficients of variation were respectively 7%, 14% and 4%.

Intermediate precision (inter run) was also evaluated on 3 control levels, and the measured coefficients of variation were respectively 10%, 13% and 14%.

Accuracy was evaluated on 3 control levels and the measured biases were respectively 1%, 2% and 0.2%.

Reproducibility was evaluated on these three levels as well as the positive control included in each series (title=490 ng/ml). The evaluation of reproducibility was based on 12 measurements over a period of 10 months. The coefficients of variation were respectively 12%, 15%, 16% and 14%.

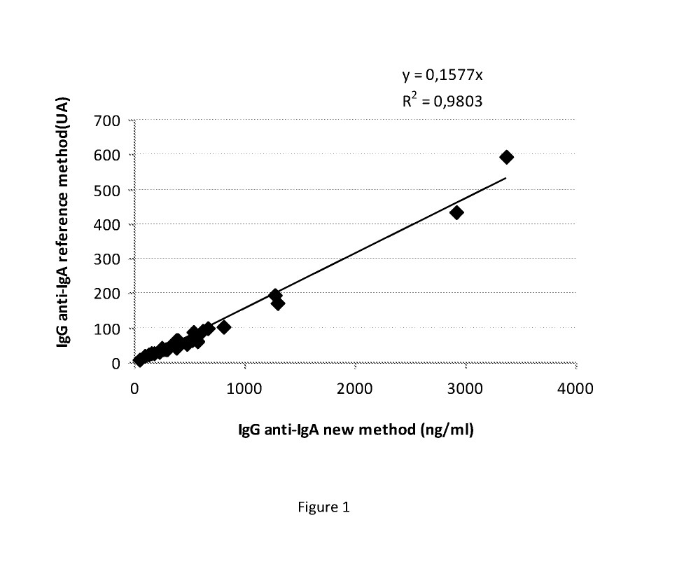

The method was well correlated with the routinely used reference method (see FIG. 1).

No interference was observed with hemolytic, lipemic or icteric samples. No cross-reactivity was seen with rheumatoid factor (high, medium and low) and monoclonal Ig (IgG kappa 17 g/L, IgG lambda 35 g/L and 15 g/L, IgA lambda 37 g/L, IgM kappa 6 g/L, cryoglobulinemia type II).

### 2) Example 2

### Quantification of Serum or Plasma Anti Factor H Auto-Antibodies Concentration

Factor H auto-antibodies are directed against Factor H, a complement alternative pathway regulatory protein. The presence of autoantibody directed against Factor H has been reported mainly in the context of atypical hemolytic uremic syndrome and glomerulonephritis. Antibodies developed in the context of allo-immunization of a patient with a complete deficiency of factor H were also observed. Finally anti-factor H antibodies have been associated with early stage of non-small cell lung cancer. The assay currently used in routine is an ELISA which requires the use of a human standard derived from Plasma exchange products from patients positive for anti-Factor H antibody. As already stated above, this method results in problems of conservation and stock depletion of the standards as well as problems relating to ethics.

2.1) Materials and Methods

50 μl of purified human factor H (Calbiochem) diluted in PBS at 10 μg/mL are coated in wells of a microplate. After saturation step, comprising the addition of 200 μl/well of PBS 0.1% Tween 20, during one hour at room temperature, individual samples diluted 1:50 in PBS 0.1% Tween 20, are performed. A 7 points standard curve is established using a murine anti factor H monoclonal antibody (OX24) applied in doubling dilution from 1/250 until the dilution 1:16000 in PBS, 0.1% Tween 20. Positive and negative controls and a well with only the buffer (blank) are also assayed. After incubation and washes, a solution of G protein conjugated with peroxidase (Hpr-protein G from GenScript; catalog product number M00090), diluted at 1:6000 in PBS, 0.1% Tween 20, is mixed to the above described composition. After washes, an enzyme substrate is applied resulting in a peroxidation reaction coloring the solution and allowing quantification by spectrophotometry of the auto-antibodies anti-factor H captured in the wells, as previously described.

The validation procedure of the assay was based on the recommendations from the COFRAC (GTA SH 04).

The evaluation of the limit of detection and limit of quantification, linearity, as well as the determination of biological reference interval, repeatability, intermediate precision, accuracy and intra-laboratory reproducibility were examined. We also studied the correlation with the reference method used in the laboratory by dosing in parallel 60 samples containing varying concentrations of anti-Factor H (20-3015 ng/ml).

The reference method used herein relies upon the same method as above, with only minor modifications. The standard curve is performed using a product of plasma exchange from one positive patient, with serial 1:2 dilutions in PBS, 0.1% Tween 20 from 1:100 to 1:3200 (6 points). The revelation antibody is a murine anti-human IgG labeled with HRP diluted at 1:500 in PBS, 0.1% Tween 20 (Sigma).

2.2) Results

The measuring range was between 20 and 480 ng/mL (as defined by the limit of quantification and the maximum of the reference curve). Lower limit of detection was 6 ng/ml. Samples were diluted to 1:50. If necessary, we additional dilutions were performed. The positivity threshold was determined at 28 ng/ml. We evaluated repeatability of 3 control levels (High=354 ng/ml, mean=106 ng/ml and low=55 ng/ml). The coefficients of variation were respectively 4%, 10% and 21%.

Intermediate precision (inter run) was evaluated on 3 control levels, and the measured coefficients of variation were respectively 17%, 16% and 26%. Overall, the method is statistically relevant as the average intermediate precision is below 20%

Accuracy of the ELISA method according to the invention was evaluated on 3 control levels and the measured biases were respectively −9%, 0% and 5%.

Reproducibility was evaluated on these three levels as well as the positive control included in each series (title=1638 ng/ml). The evaluation of reproducibility was based on six measurements over a period of 2 months. The coefficients of variation were respectively 15%, 13%, 31% and 16%.

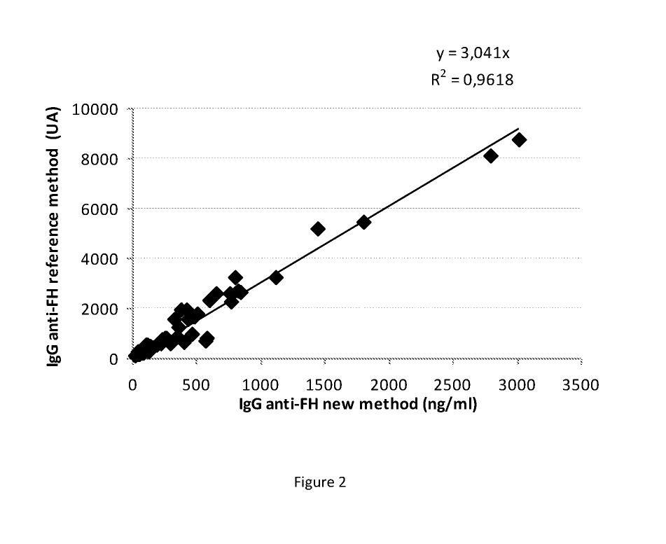

The ELISA method according to the instant invention was found statistically relevant and was well correlated with the routinely used reference method (see FIG. 2).

No interference was observed with hemolytic, lipemic or icteric samples. No cross-reactivity was seen with rheumatoid factor (high, medium and low titers) and human monoclonal Ig (IgG kappa 17 g/L, IgG lambda 35 g/L and 15 g/L, IgA lambda 37 g/L, IgM kappa 6 g/L, cryoglobulinemia type II).

### 3) Example 3

### Quantification of Plasma Eculizumab (Therapeutic Antibody) Concentration

Eculizumab is a hybrid therapeutic monoclonal antibody composed by mouse CDR regions on a structure of human IgG2 (light chains) and IgG4 (high chains). Eculizumab binds the protein C5 of the complement system and blocks its cleavage in C5b and C5a by the C5 convertases when the complement system is activated. The consequence is the absence of generation of the anaphylatoxin C5a which is implicated in inflammation and of the membrane attack complex (MAC) C5b9 involved in cellular destruction.

This treatment is recognized for diseases mediated by complement activation, and currently 2 diseases have been approved Paroxystic Nocturnial Hemoglobinuria (AMM since June 2007) and atypical hemolytic uremic Syndrome (AMM November 2011) several other indications are currently evaluated (organ graft acute rejection, autoimmune diseases).

This treatment induces a complete complement activation blockage. This is measured by the CH50 (Complement hemolytic 50). However, this test has experimental limitations due to pre-analytic conditions which high impact on the results or to the assays used, some of them having less sensibility than others. In consequence, false low levels of CH50 may be observed, impairing the good treatment adaptation. For this purpose, a more reliable dosage is necessary to monitor this particularly expensive treatment. We propose to measure free circulating eculizumbab, i.e. in excess, and not bound to the target protein.

To date, the drug is only available for therapeutic purpose and may not be used as diagnostic tools.

3.1) Materials and Methods

a) Validation of the ELISA Method According to the Invention

Diluted C5 protein, at a concentration of 5 μg/mL in PBS (Calbiochem), is coated in the wells of a microplate in order to get 50 μl/well. After a saturation step, performed by the addition of 200 μl/well of PBS with 1% BSA, for 1 hour, at 37° C., patients diluted samples (1:2000 and 1:4000) are applied as well as 5 serial dilutions of a monoclonal anti-C5 antibody (Quidel, ref A217, at a dilution of 1:1000 and then a 1:2 serial dilutions.

A positive control, namely monoclonal anti-C5 antibody, different from the one used as standard (Hycult, Ref 557), diluted at 1:4000, a negative control, which results from pooling 100 normal human plasma, and a blank point, which represents the dilution buffer are also assessed as above described. After incubation for 1 hour, at room temperature, and washes, a solution of 1:1500 diluted protein G labeled with horse radish peroxydase is added (Hpr-protein G from GenScript; catalog product number M00090). After incubation and washes, the HRP substrate is added, and the resulting colorimetric reaction is quantified as previously described

The validation procedure of the test was based on the recommendations from the COFRAC (GTA SH 04).

The limit of detection and limit of quantification, linearity, as well as the determination of biological reference interval, repeatability and intra-laboratory reproducibility were evaluated. Free eculizumab was the quantified in treated patients' plasma (PNH and aHUS) and correlated it to CH50 measured in the same samples.

b) In Vitro Determination of the Plasma Dose of Eculizumab to Inhibit C5 Activity

Between 5 to 9 plasma samples from 29 patients having Paroxystic Nocturnial Hemoglobinuria were collected and analyzed as following.

Increasing amounts of eculizumab were added in the plasma sample, i.e. from 0 to 350 μg/ml. The mixture has been incubated in a 37° C. water-bath during one hour. The samples were then diluted 1/2000 in order to be processed within an ELISA assay as described above.

Inhibition of C5 by eculizumab was assessed by measuring the CH50% as described in Costabile (Measuring the 50% Haemolytic Complement (CH₅₀) Activity of Serum. J Vis Exp. 2010; (37): 1923).

In parallel, for each dose of eculizumab added, free plasma eculizumab was assessed with either a classical ELISA method or by an ELISA method according to the invention (see a) above).

3.2) Results

a) Statistical Relevance of the ELISA Method According to the Invention

No interference was observed with hemolytic, lipemic or icteric samples. No cross-reactivity was seen with rheumatoid factor (high and low titers) and human monoclonal Ig (IgG kappa at 24 g/L, IgA kappa at 9 g/L, IgM lambda at 7 g/L and cryoglobulinemia type I).

The measuring range was between 100 and 2500 μg/mL (defined by the limit of quantification and the maximum of the reference curve), with a lower limit of detection of 30 μg/ml in the plasma. As a 1/2000 dilution of the plasma is performed before the ELISA assay, the measuring range within the sample was determined to vary between 0.050 μg/ml and 1.25 μg/ml.

Repeatability on 4 control levels (very high=4327 μg/ml, high=1086 μg/mL, medium=599 μg/ml and low=311 μg/ml) was evaluated. The coefficients of variation were respectively 3%, 3%, 3% and 7%.

Intermediate precision (inter run) was evaluated these 4 control levels, and the measured coefficients of variation were respectively 16%, 14%, 17% and 14%.

Accuracy of the ELISA method according to the invention was evaluated on the same 4 control levels and the measured biases were respectively −4%, 3%, −2% and −14%.

Reproducibility was evaluated on these four levels as well as on the positive control included in each series (title=660 μg/mL). The evaluation of reproducibility was based on 10 to 15 measurements over a period of 8 months. The coefficients of variation were respectively 14%, 17%, 14% and 16%, as well as 13% for the positive control.

625 measurements of plasmatic free eculizumab were performed in samples from 42 treated patients (6 to 40 serial samples per patient). For 7 out them, we tested also samples collected before treatment administration. No detectable eculizumab was found in these samples (<30 μg/mL). In the others, the concentrations of free eculizumab ranged from 93 to 17 460 μg/ml.

b) Determination of the Dose of Eculizumab to Efficiently Inhibit C5 Activity In Vitro

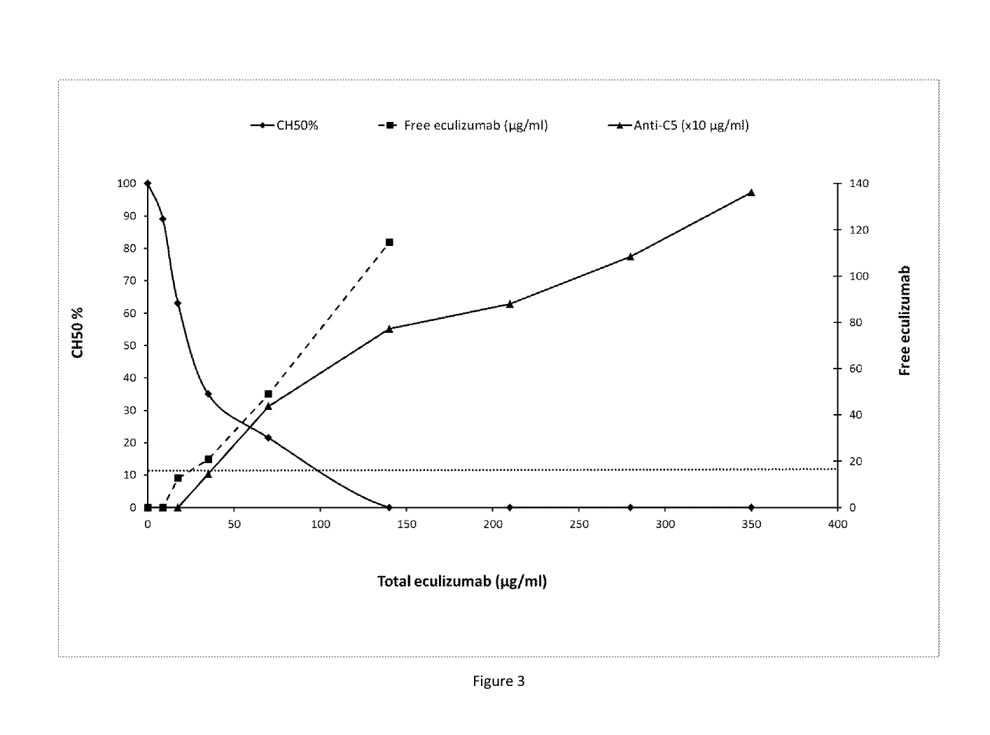

FIG. 3 illustrates the inhibition of C5 activity, as measured by CH50%, and the measure of free eculizumab for each amount of eculizumab added to the plasma sample.

As can be seen in FIG. 3, for an amount of eculizumab added in the plasma sample up to 60 μg/ml, the CH50% decreases sharply and the free plasma eculizumab increases linearly. For an amount of eculizumab above 60 μg/ml, the inhibition of C5 activity decreases slowly whereas the amount of free eculizumab still increases linearly, as measured by both the classical ELISA method (squares) or the ELISA method according to the invention (triangles).

It is to be noted that up to 75 μg/ml of eculizumab added in the plasma sample, the measure of free plasma eculizumab by either the classical ELISA method or the ELISA method according to the invention are very similar. This assay validates further the ELISA method of the invention.

It is reminded that an equilibrium is needed, in which the administration of a drug up to a certain dose needs to result in a physiological clinical benefit for the patient. In this case, the administration of 100 μg/ml of eculizumab represents an optimal dose, and results in an inhibition of 90% of the C5 activity, which is physiologically and clinically relevant. Administration of increasing amount of eculizumab only results in a limited gain of the C5 activity inhibition.

Hence, FIG. 3 illustrates that the ELISA method according to the invention is suitable for the determination of eculizumab content in the plasma of a patient.

### 4) Conclusions on Examples 1 to 3

The methods described herein allow the calibration of an ELISA assay for subsequent quantification of a target-specific test antibody. The methods described herein also provide an ELISA assay for the quantification of a target-specific test antibody. Examples 1 through 3 clearly demonstrate that the claimed methods are (i) highly specific, since no cross-reactivity of the target-specific murine calibration antibodies towards other target could be observed; (ii) highly sensitive, since the threshold of detection of allo-antibodies and auto-antibodies are above 30-50 ng/ml; and (iii) highly reproducible, since the variation between assays are ranging from 14.25% to 18.75% (lower and upper means respectively).

Hence, it is provided herein standardized methods that may be used in order to detect and quantify a target-specific test antibody that may be present in a test sample.

1. An in vitro method for quantifying a target-specific test antibody in a test sample, comprising the steps of:
a) performing an immunoassay using a target immobilized on a support which is brought into contact with the test sample, the immunoassay comprising a step of measuring the binding of the target-specific test antibody to the immobilized target by using a detectable non-antibody ligand that binds to the Fc region or a to light chain of an antibody, whereby a concentration-related value of the target-specific test antibody in the test sample is obtained, and
b) comparing the concentration-related value obtained at step a) with a reference value obtained by performing an immunoassay using the target immobilized on a support which is brought into contact with a calibration sample comprising a known concentration of a target-specific calibration antibody, the immunoassay comprising a step of measuring the binding of the target-specific calibration antibody to the immobilized target by using the detectable non-antibody ligand of step a),
and wherein:
(i) the target-specific test antibody of step a) and the target-specific calibration antibody of step b) are identical, or
(ii) the target-specific test antibody of step a) and the target-specific calibration antibody of step b) are distinct.

2. The in vitro method according to claim 1, wherein:
(i) the target-specific test antibody of step a) and the target-specific calibration antibody of step b) originate from the same animal species, or
(ii) the target-specific test antibody of step a) and the target-specific calibration antibody of step b) originate from distinct animal species.

3. The in vitro method according to claim 1, wherein each of (i) the target-specific test antibody and (ii) the target-specific calibration antibody is independently selected in a group comprising human and non-human antibodies.

4. The in vitro method according to claim 3, wherein said non-human antibody is selected in a group comprising mammal antibodies, avian antibodies, amphibian antibodies, reptile antibodies, fish antibodies and insect antibodies.

5. The in vitro method according to claim 1, wherein the target-specific test antibody is a human antibody.

6. The in vitro method according to of claim 1, wherein the target-specific test antibody is selected in a group comprising auto-antibodies, allo-antibodies, therapeutic antibodies, imaging antibodies.

7. The in vitro method according to of claim 1, wherein the target-specific test antibody is a human antibody and the target-specific calibration antibody is a non-human antibody, preferably selected in a group comprising mouse antibodies, rat antibodies, llama antibodies, goat antibodies, sheep antibodies, rabbit antibodies and horse antibodies, and is preferably a mouse antibody.

8. The in vitro method according to claim 1, wherein the light chain consists in a kappa light chain.

9. The in vitro method according to claim 1, wherein said detectable non-antibody ligand is selected in a group comprising protein A, protein G, protein A/G, protein L and is preferably protein G.

10. The in vitro method according to claim 1 the detectable non-antibody ligand is labelled with a detectable molecule.

11. The in vitro method according to claim 10, wherein the detectable molecule is selected in a group comprising a radioactive molecule, a chemo-luminescent molecule, a fluorescent molecule, a fluorophore and an enzyme.

12. The in vitro method according to claim 1, wherein the test sample is selected in a group comprising a blood sample, a plasma sample, a serum sample, a lymph sample, a cerebrospinal fluid sample, an urine sample and a milk sample.

13. The in vitro method according to claim 1, wherein the immunoassay is an ELISA.

14. A kit for quantifying a target-specific antibody in a test sample, comprising:
a target-specific calibration antibody, and;
a detectable non-antibody ligand that binds to the Fc region or to a light chain of an antibody.

15. The kit according to claim 14, which further comprises the target.

16. The kit according to claim 14, which further comprises one or more reagents for detecting the non-antibody ligand.

17. The kit according of claim 14, wherein the detectable non-antibody ligand is selected in a group comprising protein A, protein G, protein A/G and protein L.
